(19) United States
(12) Patent Application Publication
(10) Pub. No.: US 2026/0284064 A1
(43) Pub. Date: Sep. 24, 2026

(54) PI3K INHIBITORS FOR THE TREATMENT OF CANCER

(71) Applicant: Leapfrog Bio, Inc., South San Francisco, CA (US)

(72) Inventor: Tomas BABAK, Oakland, CA (US)

(21) Appl. No.: 19/474,613

(22) PCT Filed: Apr. 10, 2024
(86) PCT No.: PCT/US2024/023930

§ 371 Date: Oct. 10, 2025

Related U.S. Application Data
(60) Provisional application No. 63/495,530, filed on Apr. 11, 2023.

(51) Int. Cl. A61K 31/5377; A61P 35/00

(52) U.S. Cl. CPC A61K 31/5377; A61P 35/00

(57) ABSTRACT

Provided herein are methods of using a PI3K inhibitor in the treatment of a cancer comprising a loss of function mutation or a deletion of a PI3K gene.

### CROSS REFERENCE TO RELATED APPLICATIONS

This application claims the benefit and priority of U.S. Provisional Application No. 63/495,530 filed on Apr. 11, 2023, the entirety of which is incorporated herein by reference.

### BACKGROUND

The Phosphoinositide 3-kinase (PI3K) pathway may play an important role in many biological processes, including cell cycle progression, cell growth, survival, actin rearrangement and migration, and intracellular vesicular transport. PI3Ks are heterodimers consisting of a p110 catalytic subunit and a p85 regulatory subunit. In mammals, there are four genes, PIK3CA, PIK3CB, PIK3CD, and PIK3CG encoding p110 catalytic isoforms: p110-alpha (also referred to as PI3K-alpha), p110-beta, p110-delta, and p110-gamma respectively. PI3K activation includes generation of phosphatidylinositol-3,4,5-trisphosphate (PI(3,4,5)P3) by the activated catalytic subunit, which in turn activates multiple downstream signaling events through AKT, MTOR, various transcription factors, and other signaling components. The concept of targeting specific PI3K isoforms with isoform selective inhibitors is actively being explored in cancer research. Although PI3K inhibitors are being research for use in the treatment of cancer, resistance to PI3K inhibitors remains a significant challenge. Accordingly, there is a need for the development of novel PI3K inhibitor treatments for cancer-targeted therapies.

### BRIEF SUMMARY OF THE INVENTION

The present disclosure is based, in part, on the discovery that cancers may be associated with a PIK3CA loss of function genetic mutation, and once identified, such cancers may be treated by administering (e.g., a therapeutically effective amount) a PI3K inhibitor. Provided herein, in some embodiments, is a method of treating a cancer in a patient in need thereof, said method comprising: administering a composition comprising a phosphoinositide 3-kinase (“PI3K”) inhibitor; wherein said cancer has been previously determined as comprising a loss of function mutation or a deletion of a PIK3CA gene. In some embodiments, said loss of function or deletion of said PIK3CA gene reduces an amount or function of PI3K-alpha as compared to a wild-type amount or function of PI3K-alpha.

Further provided herein, in some embodiments, is a method of treating a cancer in a patient in need thereof, said method comprising: administering a composition comprising a phosphoinositide 3-kinase (“PI3K”) inhibitor; wherein said patient has a reduced amount or function of PI3K-alpha as compared to a wild-type amount or function of PI3K-alpha. In some embodiments, said patient has been previously determined as comprising a reduced amount or function of PI3K-alpha as compared to a wild-type amount or function of PI3K-alpha. In some a mutation or deletion in a gene reduces said amount of function of PI3K-alpha (e.g., such as found in a non-cancerous corresponding cell). In some embodiments, said gene is a PIK3CA gene. In some embodiments, said mutation is a loss of function or deletion of said gene. In some embodiments, a cancer (e.g., cancer cell) has said reduced amount of function of PI3K-alpha. In some embodiments, said PI3K inhibitor is a PI3K-beta inhibitor. In some embodiments, said PI3K-beta inhibitor is GSK-2636771, AZD-6482, AZD-8186, KA-2237, SAR-260301, TGX-221, or a combination thereof. In some embodiments, said PI3K-beta inhibitor is a selective PI3K-beta inhibitor. In some embodiments, said selective PI3K-beta inhibitor is GSK-2636771, AZD-6482, AZD-8186, KA-2237, TGX-221, or SAR-260301. In some embodiments, said selective PI3K-beta inhibitor is GSK-2636771. In some embodiments, said PI3K-beta inhibitor is AZD-6482. In some embodiments, said PI3K-beta inhibitor is AZD-8186. In some embodiments, said PI3K-beta inhibitor is KA-2237. In some embodiments, said PI3K-beta inhibitor is SAR-260301. In some embodiments, said PI3K-beta inhibitor is TGX-221. In some embodiments, said administering of said PI3K-beta inhibitor decreases an adverse effect or a side effect compared to a corresponding administering of a pan-PI3K inhibitor, a PI3K-alpha inhibitor, a PI3K-gamma inhibitor, or a PI3K-delta inhibitor to said cancer. In some embodiments, said administering increases cancer cell inhibition compared to a corresponding administration of said composition to a second patient comprising a wild-type amount or function of PI3K-alpha.

Further provided herein, in some embodiments, are methods of treating a cancer in a patient in need thereof, said method comprising: administering to said patient a composition comprising a PI3K-beta inhibitor, wherein when said composition is administered, cancer cell inhibition increases compared to a corresponding composition comprising a PI3K inhibitor other than said PI3K-beta inhibitor. In some embodiments, said PI3K-beta inhibitor is GSK-2636771, AZD-6482, AZD-8186, KA-2237, TGX-221, or SAR-260301. In some embodiments, said PI3K-beta inhibitor is a selective PI3K-beta inhibitor. In some embodiments, selective PI3K-beta inhibitor is GSK-2636771, AZD-6482, AZD-8186, KA-2237, TGX-221, or SAR-260301. In some embodiments, said PI3K-beta inhibitor is an inhibitor of PI3K-beta and PI3K-delta. In some embodiments, said PI3K-beta inhibitor is AZD8186 or KA-2237. In some embodiments, said cancer comprises breast cancer, urothelial cancer, rectal cancer, thymus cancer, sarcoma, pancreatic cancer, or a combination thereof. In some embodiments, said urothelial cancer comprises bladder cancer.

Further provided herein, in some embodiments, are kits for treating a cancer, comprising: a test for determining either: a loss of function mutation or a deletion of a PIK3CA gene; a reduced amount or function of PI3K-alpha as compared to a wild-type amount or function of PI3K-alpha; and a phosphoinositide 3-kinase (“PI3K”) inhibitor. In some embodiments, said PI3K inhibitor is a PI3K-beta inhibitor. In some embodiments, said PI3K-beta inhibitor is GSK-2636771, AZD-6482, AZD-8186, KA-2237, SAR-260301, TGX-221, or a combination thereof.

In some embodiments, said PI3K-beta inhibitor is a selective PI3K-beta inhibitor. In some embodiments, said selective PI3K-beta inhibitor is GSK-2636771, AZD-6482, AZD-8186, KA-2237, TGX-221, or SAR-260301. In some embodiments, said cancer comprises breast cancer, urothelial cancer, rectal cancer, thymus cancer, sarcoma, pancreatic cancer, or a combination thereof. In some embodiments, said urothelial cancer comprises bladder cancer.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates proteomic levels of various markers that capture the activity of PI3K signaling in breast cancer.

FIG. 2A-2C illustrate the activity of PI3K inhibitors in KP4 cells with and without a loss of function mutation in a PIK3CA gene. FIG. 2A demonstrates the activity of AZD-8186, a PI3K inhibitor with inhibitive activity against PI3K-beta and PI3K-delta. FIG. 2B demonstrates the activity of GSK-2836771, a PI3K inhibitor with inhibitive activity against PI3K-beta. FIG. 2C demonstrates the activity of seletalisib, a PI3K inhibitor with inhibitive activity against PI3K-delta.

FIG. 3A-3D illustrate the activity of PI3K inhibitors in KP4 cells with and without PIK3CA gene knocked out. FIG. 3A demonstrates the activity of SAR-260301, a PI3K inhibitor with inhibitive activity against PI3K-beta. FIG. 3B demonstrates the activity of AZD-6482, a PI3K inhibitor with inhibitive activity against PI3K-beta. FIG. 3C demonstrates the activity of Acalisib, a PI3K inhibitor with inhibitive activity against PI3K-delta. FIG. 3D demonstrates the activity of TGX-221, a PI3K inhibitor with inhibitive activity against PI3K-beta.

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The present disclosure provides for new methods of treatment or prevention of cancers, resulting in unexpectedly superior cancer cell inhibition or cancer treatment. The methods provided herein may utilize synthetic lethality to overcome challenges associated with PI3K inhibitors, such as the development of resistance to PI3K inhibitors.

Synthetic lethality is a phenomenon in which the simultaneous loss of function or deletion (or a combination of both) of two genes together leads to cell death, whereas the loss of function of either gene alone does not lead to cell death. This concept may be used in cancer research as a therapeutic strategy for target identification, where a goal is to identify pairs of genes whose combined inhibition leads to cell death in cancer cells while not resulting in the death of non-cancerous cells. An important concept underlying synthetic lethality may be to target a vulnerability in cancer cells that is not present in normal cells. Since cancer cells already have a mutated gene, a promising approach may be to identify and then pharmacologically target a synthetically lethal partner of one of the mutated genes. However, the identification of synthetic lethal gene pairs and the further identification of a pharmaceutical intervention for use in targeting the synthetically lethal partner remains a significant challenge.

In particular embodiments, the present disclosure provides new methods and kits for treating a cancer through synthetic lethality. Synthetic lethality may occur with cancers comprising a loss of function mutation in the PIK3CA gene or a deletion of the PIK3CA by administration of a PI3K inhibitor. In some embodiments, the method comprises treating a cancer comprising a loss of function mutation in a PI3K gene or a deletion of a PI3K gene. In some embodiments, the loss of function mutation is in a PIK3CA gene. In some embodiments, the deletion is of the PIK3CA gene. In some embodiments, the PI3K inhibitor comprises a PI3K-beta inhibitor.

In some embodiments, the present disclosure provides methods of treating or reducing the incidence of a cancer, said method comprising administering said composition to a subject in need thereof, wherein said composition comprises a phosphoinositide 3-kinase (“PI3K”) inhibitor. In some embodiments, said cancer has a reduced amount or function of PI3K-alpha (e.g., as compared to a wild-type amount or function of PI3K-alpha). In some embodiments, said cancer has been previously determined as comprising a reduced amount or function of PI3K-alpha as compared to a wild-type amount or function of PI3K-alpha. The reduced amount or function of PI3K-alpha may be a result of a mutation causing or caused by the cancer.

### I. Methods of Treating Cancer

Provided herein, in some embodiments, are methods of treating a cancer in a patient in need thereof. In some embodiments, the method comprises administering (e.g., a therapeutically effective amount of) a composition, such as any of the compositions described herein, to a patient having a cancer. In some embodiments, the composition comprises a PI3K inhibitor. In some embodiments, the PI3K inhibitor is a PI3K beta inhibitor. In some embodiments, the PI3K beta inhibitor is a selective PI3K beta inhibitor. In some embodiments, the cancer comprises a loss of function mutation or a deletion of a gene (e.g., PIK3CA gene). In some embodiments, the cancer has been determined (e.g., previously determined) as comprising a loss of function mutation in a gene or a deletion of a gene (e.g., PIK3CA gene). In some embodiments, said patient comprises a reduced amount (i.e., number) of PI3K-alpha or function of PI3K-alpha. In some embodiments, said reduced amount of PI3K-alpha or function of PI3K-alpha is in a cancer cell of the subject.

a. PI3K Inhibitors

Phosphoinositide 3-kinase (PI3K) signaling may be involved in important physiological and pathophysiological functions that drive tumor progression, such as metabolism, cell growth, proliferation, angiogenesis and metastasis. Pharmacological or genetic suppression of this signaling may yield cancer cell death or regression of tumor growth. The PI3K pathway may be activated via point mutation of the PIK3CA gene or inactivation of the phosphatase and tensin homolog (PTEN) gene. Activation of this pathway occurs in approximately 30-50% human cancers and may contribute to resistance to various anti-cancer therapies.

PI3K proteins may be classified into three main classes (I, II and III) based on the substrate specificities and structural characteristics. Class I PI3Ks may be further categorized into two subtypes (A and B) based on the mode of regulation. Class IA PI3Ks form dimers comprising a regulatory (p85a, p850, p55a, p557, p50a) and a catalytic (p110a, p110β, p110δ) subunit. Class I PI3Ks may act downstream of both G protein-coupled receptors (GPCRs) and receptor tyrosine kinases (RTKs). In addition, these regulatory subunits may play an important role in stabilization of the p110 catalytic subunits and in suppression of the basal lipid kinase activity. Class IB PI3Ks, which may be activated downstream of GPCRs, may form heterodimers consisting of p110γ as catalytic subunit with other regulatory subunits such as p101, p87 or p84. Class IB PI3Ks consist of three isoforms (PI3K-C2a, PI3K-C213 and PI3K-C27). These isoforms may have a RAS-binding domain (RBD), a helical domain, and a catalytic domain but may lack a regulatory domain. The class III PI3Ks, also known as vacuolar protein sorting 34 (VPS34), may heterodimerize with membrane-associated VPS15 regulatory subunit. This VPS34-VPS15 complex may be ubiquitously expressed in mammals regulating several functions such as phagocytosis, autophagy, endocytosis and intracellular trafficking. Some reports have considered mTOR as class IV PI3K-related kinases, whose catalytic domain is associated with ataxia-telangiectasia (ATM), fluorescence recovery after photo bleaching (FRAP) and transformation/transcription domain-associated protein (TRRAP) and FK506-binding protein 12 (FKBP12)/rapamycin (FRB)-binding domain and a C-terminal FAT domain (FATC). There are two Huntington elongation factor 3, PR65/A subunit of protein phosphatase 2A, TOR (HEAT) repeats in the N-terminal region which modulate the protein-protein interactions in mTOR signaling. The four described isoforms of the catalytic subunit p110α, p110β, p110γ, and p110δ, are encoded by genes PIK3CA, PIK3CB, PIK3CG, and PIK3CD, respectively.

Inhibiting compounds (e.g., small-molecule inhibitors) targeting PI3K include pan, isoform-specific, and dual PI3K/mTOR inhibitors. To date, four PI3K inhibitors (Copanlisib, Idelalisib, Duvelisib, and Alpelisib) have been approved by the United States Food and Drug Administration (FDA). However, other PI3K inhibitors are actively being researched. Provided below are non-limiting descriptions of a few exemplary PI3K inhibitors.

i. GSK-2636771

GSK-2636771 (CAS No. 1372540-25-4) is a substituted benzimidazole and is a potent, selective and orally bioavailable inhibitor of class I PI3Kβ. For p110β, it has demonstrated an IC₅₀ of 5.2 nM, a 900-fold selectivity over p110α and p110γ, and 10-fold selectivity over p110δ isoforms. A phase II clinical trial (titled “A Phase I/IIa, First Time in Human, Study of GSK-2636771 in Subjects With Advanced Solid Tumors With Phosphatase and Tensin Homolog (PTEN) Deficiency”) was underway to identify the effects of GSK-2636771 in patients whose cancer has a complete loss of PTEN expression.

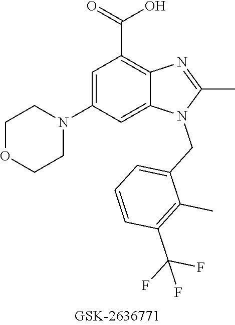 [SMILES: Cc1c(Cn2c(C)nc3c(C(=O)O)cc(N4CCOCC4)cc32)cccc1C(F)(F)F]

Provided herein, in some embodiments, is a method of treating a cancer in a patient in need thereof, said method comprising administering GSK-2636771, wherein said cancer a loss of function mutation or a deletion of a PIK3CA gene. In some embodiments, said cancer has been previously determined as comprising a loss of function mutation or a deletion of a PIK3CA gene. Further provided herein in some embodiments, is a method of treating a cancer in a patient in need thereof, said method comprising administering GSK-2636771, wherein said patient has a reduced amount or function of PI3K-alpha (e.g., as compared to a wild-type amount or function of PI3K-alpha). In some embodiments, said cancer has been previously determined as comprising a reduced amount or function of PI3K-alpha (e.g., as compared to a wild-type amount or function of PI3K-alpha).

For more information on GSK-2636771, see: Preparation of benzimidazole derivatives as PI3 kinase inhibitors, Qu, et. al., WO2012047538 A1 2012-04-12; and A First-Time-in-Human Study of GSK-2636771, a Phosphoinositide 3 Kinase Beta-Selective Inhibitor, in Patients with Advanced Solid Tumors By: Mateo, Joaquin; Ganji, Gopinath; Lemech, Charlotte; Burris, Howard A.; Han, Sae-Won; Swales, Karen; Decordova, Shaun; De Young, M. Phillip; Smith, Deborah A.; Kalyana-Sundaram, Shanker; et al Clinical Cancer Research (2017), 23(19), 5981-5992, which are herein by reference in their entirety.

ii. AZD-8186

AZD-8186 is a PI3K inhibitor, which has demonstrated potent inhibition of PI3Kβ and PI3K6. AZD-8186 has demonstrated an IC₅₀ of 4 nM, 12 nM, 35 nM, and 675 nM, for PI3Kβ, PI3K6, PI3Kα and PI3K7, respectively. In a broad panel of protein and lipid kinase assays, selectivity for PI3Kβ and 6 was >100-fold versus 74 protein and lipid kinases. At 10 M, AZD-8186 demonstrated no significant binding to 442 other kinases in a KinomeScan screen. AZD-8186 showed selectivity for PI3K family kinases and no other off-target activity was detected. In a PTEN-null line, in MDA-MB-468 cells, AZD-8186 inhibited PI3Kβ-dependent activation of pAKT (Ser473) with an IC₅₀ value of 3 nM. Potency in the PIK3CA-mutant line BT474c was 752 nM, which demonstrated selectivity for PI3Kβ over PI3Kα. IgM mediated stimulation of B cells resulted in phosphorylation of AKT through activation of PI3K6. AZD-8186 inhibited IgM-stimulated phosphorylation of pAKT (Ser473) activation in JEKO cells with an IC₅₀ value of 17 nM. In cell proliferation assays, AZD-8186 inhibited proliferation of MDA-MB-468 cells with a GI₅₀ value of 65 nM (compare to IgM stimulated JEKO cell growth with an IC₅₀ value of 228 nM). It only inhibited BT474c cell growth with an IC₅₀ value of 1.981 M, which is consistent with AZD8186's selectivity for PI3Kβ over PI3Kα.

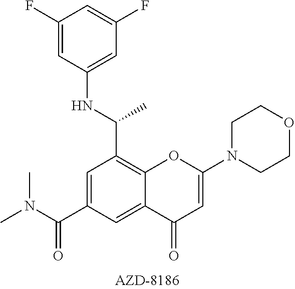 [SMILES: C[C@@H](Nc1cc(F)cc(F)c1)c1cc(C(=O)N(C)C)cc2c(=O)cc(N3CCOCC3)oc12]

To assess single-agent efficacy of AZD-8186 in vivo, antitumor activity has been assessed in the PTEN-null TNBC models HCC70 and MDA-MB-468, and the prostate models PC3 and HID28. At 50 and 25 mg/kg twice a day, AZD-8186 was shown to inhibit the growth of all four models. At 25 and 50 mg/kg, HCC70 was inhibited at 62% (P<0.001) and 85% (P<0.001), respectively, MDA-MB-468 was inhibited at 47% (P<0.001) and 76% (P<0.001), respectively, at end at of this study, with regression early in the study. Efficacy in the PTEN-null prostate model, PC3 was less pronounced with 25 and 50 mg/kg giving maximal growth inhibition of 59% (P<0.001) and 64% (P<0.001), respectively. In contrast, AZD-8186 gave 79% (P<0.001) growth inhibition in the PTEN-null prostate explant model HID28. In mouse, AZD-8186 demonstrated a short half-life delivering a PK profile that resulted in intermittent cover over 24 hours dosing interval. To increase the time of exposure, animals bearing PC3 tumors were co-dosed with AZD-8186 in the presence of the Cyt P450 inhibitor ABT, which resulted in significantly increased exposure. This was also shown to increase the efficacy in the PC3 model with 86% (P<0.005) reduction in tumor growth achieved with 30 mg/kg AZD-8186+ABT.

For more information on AZD-8186, see: Chromenone derivatives, Barlaam, et. al. U.S. Pat. No. 8,399,460; and Discovery of (R)-8-(1-(3,5-Difluorophenylamino)ethyl)-N,N-dimethyl-2-morpholino-4-oxo-4H-chromene-6-carboxamide (AZD8186): A Potent and Selective Inhibitor of PI3Kβ and PI3Kδ for the Treatment of PTEN-Deficient Cancers By: Barlaam, er. al. Journal of Medicinal Chemistry (2015), 58(2), 943-962, which are herein by reference in their entirety. ii. AZD-6482

AZD-6482 (KIN-193) is a potent and selective PI3Kβ inhibitor. Specifically, AZD-6482 is a p110β inhibitor that has demonstrated an IC50 of 0.69 nM. In vitro kinase assay AZD-6482 has demonstrated 200, 20, and 70-fold selectivity for p110β over p110α, p110δ, and p110γ isoforms, respectively. AZD-6482 may also exhibit selectivity of ~80 fold over PI3K-C2β and DNA-PK and more than 1,000-fold over other phosphatidylinositol-3 kinase-related kinases (PIKKs). An inhibitor-kinase interaction profiling of AZD-6482 against a panel of 433 kinases using the KinomeScan approach demonstrated that AZD-6482 is highly selective in its interaction with PI3Ks. AZD-6482 may selectively target PTEN-deficient tumors. When AZD-6482 was tested on cell proliferation on a large panel of 422 cancer cell lines using high-throughput tumor cell line profiling, 35% of cell lines with PTEN mutations (20 out of 57) and 16% of cell lines with wild-type PTEN (58 out of 365) were sensitive to AZD-6482 with a threshold of EC₅₀<5 μM.

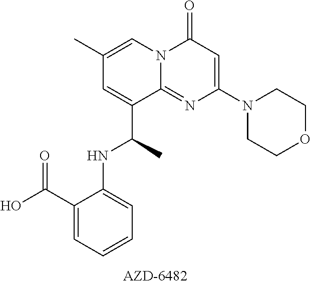 [SMILES: Cc1cc([C@@H](C)Nc2ccccc2C(=O)O)c2nc(N3CCOCC3)cc(=O)n2c1]

iv. SAR-260301

SAR-260301 is an orally available and selective PI3Kβ inhibitor. It has demonstrated an IC50 of 1539/23/469/10000 nM against PI3K α/β/δ/γ, respectively. SAR-260301 has shown promising preclinical activity against a range of cancer types, including breast, prostate, and ovarian cancers. It has been shown to inhibit PI3Kβ signaling and downstream pathways, leading to cell death in cancer cells. SAR-260301 is currently being evaluated in clinical trials for the treatment of advanced solid tumors, with a focus on breast and prostate cancers.

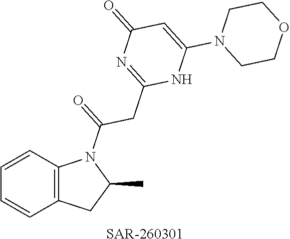

v. KA-2237

KA-2237 is an oral, potent, dual PI3Kβ and PI3K δ (p110β/p110δ) inhibitor. In preclinical studies, KA-2237 inhibited p110β- and p110δ-dependent AKT activation and suppressed proliferation of diverse hematological and epithelial tumors.

vi. Copanlisib

Copanlisib has been indicated for the treatment of adult patients with relapsed follicular lymphoma (FL) who have received at least two prior systemic therapies. Copanlisib is an inhibitor of phosphatidylinositol-3-kinase (PI3K) with inhibitory activity predominantly against PI3K-α and PI3K-6 isoforms expressed in malignant B cells. It demonstrated an IC₅₀ of 0.5, 3.7, 6.4, and 0.7 nM in cell-free assays for PI3Kα/β/γ/δ, respectively. Copanlisib has been shown to induce tumor cell death by apoptosis and inhibition of proliferation of primary malignant B cell lines. Copanlisib has been shown to inhibit several key cell-signaling pathways, including B-cell receptor (BCR) signaling, CXCR12 mediated chemotaxis of malignant B cells, and NFκB signaling in lymphoma cell lines.

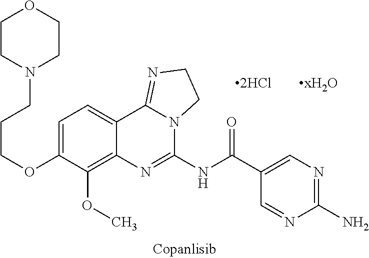 [SMILES: COc1c(OCCCN2CCOCC2)ccc2c1N=C(NC(=O)c1cnc(N)nc1)N1CCN=C21]

vii. Idelalisib

Idelalisib is an inhibitor of phosphatidylinositol 3-kinase delta (PI3K), which may be expressed in normal and malignant B-cells. It showed an IC₅₀ of 2.5 nM in cell-free assays for PI3K6, where the selectivity for PI3Kδ was 40- to 300-fold greater than PI3Kα/β/γ. Idelalisib has been shown to induce apoptosis and inhibit proliferation in cell lines derived from malignant B-cells and in primary tumor cells. Idelalisib also inhibited several cell signaling pathways, including B-cell receptor (BCR) signaling and CXCR4 and CXCR5 signaling, which are involved in trafficking and homing of B-cells to the lymph nodes and bone marrow. Treatment of lymphoma cells with idelalisib has been shown to inhibit chemotaxis and adhesion, and reduce cell viability.

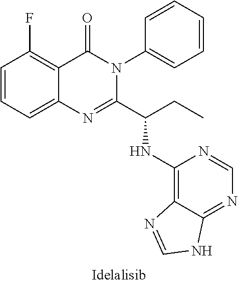 [SMILES: CC[C@H](Nc1ncnc2[nH]cnc12)c1nc2cccc(F)c2c(=O)n1-c1ccccc1]

viii. Duvelisib

Duvelisib is indicated for the treatment of adult patients with relapsed or refractory chronic lymphocytic leukemia (CLL) or small lymphocytic lymphoma (SLL) after at least two prior therapies. Duvelisib is an inhibitor of PI3K with inhibitory activity predominantly against PI3K-δ and PI3K-γ isoforms, which may be abnormally expressed in normal and malignant B-cells. Duvelisib demonstrated an IC₅₀ of 1 nM for PI3K-δ and 50 nM for PI3K-γ in cell-free assays. Duvelisib has been shown to induce growth inhibition and reduce viability in cell lines derived from malignant B-cells and in primary CLL tumor cells. Duvelisib was shown to inhibit several key cell-signaling pathways, including B-cell receptor signaling and CXCR12-mediated chemotaxis of malignant B-cells. Additionally, duvelisib inhibited CXCL12-induced T cell migration and M-CSF and IL-4 driven M2 polarization of macrophages.

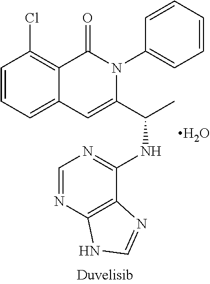

ix. Alpelisib

Alpelisib is an inhibitor of PI3K with inhibitory activity predominantly against PI3Kα. Alpelisib is indicated, in combination with fulvestrant, for the treatment of postmenopausal women, and men, with hormone receptor (HR)-positive, human epidermal growth factor receptor 2 (HER2)-negative, PIK3CA mutated, advanced or metastatic breast cancer as detected by an FDA-approved test following progression on or after an endocrine-based regimen. It showed IC50 of 5 nM in a cell-free assay for PI3Kα, and minimal effect on PI3Kβ/γ/δ.

In studies, gain-of-function mutations in the gene encoding the catalytic α-subunit of PI3K (PIK3CA) led to activation of PI3Kα and Akt-signaling, cellular transformation and the generation of tumors in in vitro and in vivo models. In breast cancer cell lines, alpelisib inhibited the phosphorylation of PI3K downstream targets, including Akt, and showed activity in cell lines harboring a PIK3CA mutation. In vivo, alpelisib inhibited the PI3K/Akt signaling pathway and reduced tumor growth in xenograft models, including models of breast cancer. PI3K inhibition by alpelisib treatment has been shown to induce an increase in estrogen receptor (ER) transcription in breast cancer cells. The combination of alpelisib and fulvestrant demonstrated increased antitumor activity compared to either treatment alone in xenograft models derived from ER-positive, PIK3CA mutated breast cancer cell lines.

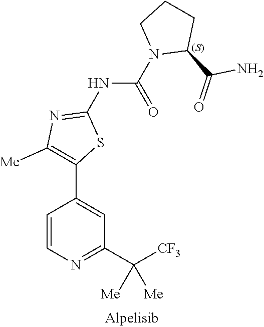

x. Seletalisib

Seletalisib (UCB5857) is potent and selective PI3Kδ inhibitor with an IC50 of 12 nM for PI3K6. In studies, Seletalisib has been shown to block AKT phosphorylation following activation of the BCR in a B-cell line. Seletalisib inhibited N-formyl peptides (fMLP)-stimulated, but not phorbol myristate acetate (PMA)-stimulated superoxide release from human neutrophils consistent with a PI3Kδ-specific activity. No indications of cytotoxicity were observed in PBMCs or other cell types treated with seletalisib. Seletalisib was shown to block human T-cell production of several cytokines from activated T-cells. Seletalisib was also shown to inhibit T-cell differentiation to Th1, Th2, and Th17 subtypes. Additionally, seletalisib inhibited B-cell proliferation and cytokine release. In human whole blood assays, seletalisib inhibited CD69 expression upon B-cell activation and anti-IgE-mediated basophil degranulation.

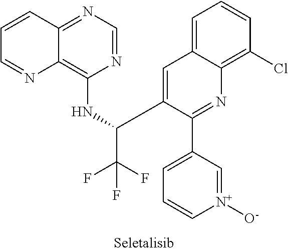 [SMILES: [O-][n+]1cccc(-c2nc3c(Cl)cccc3cc2[C@@H](Nc2ncnc3cccnc23)C(F)(F)F)c1]

For more information on Seletalisib, see: WO2012032334 A1 2012-03-15; and European Journal of Clinical Pharmacology (2017), 73(5), 581-591, which are herein by reference in their entirety.

xi. Acalisib

Acalisib (GS-9820) is a potent and selective PI3Kδ inhibitor. Acalisib has been reported to have IC50 values as low 12.7 and 14 nM against PI3Kδ, and has shown to have approximately greater than 100 fold selectivity over the other class 1 PI3K enzymes. Acalisib has demonstrated an IC50 of 3377 against PI3Kβ. It is suggested that Acalisib may not have activity against Class II and III PI3K family members.

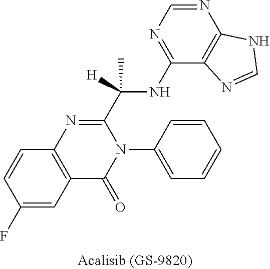 [SMILES: C[C@H](Nc1ncnc2[nH]cnc12)c1nc2ccc(F)cc2c(=O)n1-c1ccccc1]

xii. TGX-221

TGX-221 is a potent and selective PI3Kβ inhibitor. Specifically, TGX-221 has demonstrated activity as a p110β inhibitor. TGX-221 has been reported to have IC50 values as low 5 nM for PI3Kβ, and has shown to have approximately greater than 1000 fold selectivity over the other PI3K enzymes (e.g., PI3Kα). Additionally, TGX-221 has been shown to have high cell permeability.

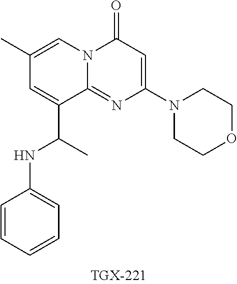 [SMILES: Cc1cc(C(C)Nc2ccccc2)c2nc(N3CCOCC3)cc(=O)n2c1]

b. PI3K Beta Inhibitor

In some embodiments, the methods provided herein comprise administering a PI3K inhibitor, such as, for example, any of the PI3K inhibitors disclosed herein. In some embodiments, the PI3K inhibitor comprises a pan-PI3K inhibitor. In some embodiments, the PI3K inhibitor comprises a PI3K beta inhibitor.

In some embodiments, the PI3K beta inhibitor comprises GSK-2636771, AZD-6482, AZD-8186, KA-2237, SAR-260301, ACP-319, Alpelisib, Apitolisib, BAY-1082439, BGT-226, Bimiralisib, Copanlisib, Dactolisib, Duvelisib, Fimepinostat, GSK-2126458, NMEN-1611, OP-11, PF-4691502, Pictilisib, Samotolosib, Taselisib, TG-100-115, or VS-5584.

The PI3K inhibitor may selectively inhibit PI3K beta or may inhibit PI3K beta along with one or more other PI3K isoforms (e.g., PI3K delta). In some embodiments, the PI3K inhibitor comprises a PI3K beta inhibitor and a PI3K inhibitor of one or more of the other isoforms of PI3K (e.g., a PI3K alpha inhibitor, a PI3K gamma inhibitor, a PI3K delta inhibitor, or a combination thereof). In some embodiments the PI3K inhibitor comprises a PI3K beta inhibitor and a PI3K delta inhibitor. In some embodiments, the PI3K beta inhibitor and PI3K delta inhibitor comprises AZD8186, or KA-2237.

In some embodiments, the PI3K beta inhibitor is a selective PI3K beta inhibitor. A selective PI3K beta inhibitor may target the PI3K beta isoform with at least a 3× higher selectivity for the PI3K beta isoform (e.g., IC50 binding specificity that is 3× or more lower, or IC50 cell viability that is 3× or more lower) compared to other PI3K isoforms. For example, the selective PI3K beta inhibitor may inhibit PI3K beta, but provide minimal or no inhibition against PI3K alpha, PI3K gamma, or PI3K delta. An example of a selective PI3K beta inhibitor is GSK-2636771, which has demonstrated an IC₅₀ of 5.2 nM for p110β, showing 900-fold selectivity over p110α and p110γ, and 10-fold selectivity over p110δ isoforms. As a further example, AZD 6482, a selective PI3K beta inhibitor, has demonstrated an IC50 of 0.69 nM for p110β, a 200, 20, and 70-fold selectivity for p110β over p110α, p110δ, and p110γ isoforms, respectively. Furthermore, SAR-260301, a selective PI3K beta inhibitor, has demonstrated an IC50 of 1539/23/469/10000 nM against PI3K α/β/δ/γ, respectively.

In some embodiments, the PI3K beta inhibitor comprises a selective PI3K beta inhibitor. In some embodiments, the selective PI3K beta inhibitor comprises GSK-2636771, AZD-6482, AZD-8186, KA-2237, AZD-8186, KA-2237, or SAR-260301. In some embodiments, the selective PI3K beta inhibitor comprises GSK-2636771, AZD-6482, or SAR-260301. In some embodiments, the selective PI3K beta inhibitor comprises GSK-2636771. In some embodiments, the selective PI3K beta inhibitor comprises AZD-6482. In some embodiments, the selective PI3K beta inhibitor comprises KA-2237. In some embodiments, the selective PI3K beta inhibitor comprises AZD-8186. In some embodiments, the selective PI3K beta inhibitor comprises SAR-260301.

The utilization of a selective PI3K beta inhibitor may be useful in treating a cancer, such as those disclosed herein (e.g., those cancers comprising a loss of function mutation in PIK3CA or a deletion of PIK3CA), while potentially lowering the incidence or severity of adverse effects, associated with the treatment.

c. PIK3CA Gene

PI3K may add a cluster of oxygen and phosphorus atoms (a phosphate group) to other proteins through phosphorylation. PI3K phosphorylates certain signaling molecules, which may trigger a series of additional reactions that transmit chemical signals within cells. PI3K signaling may be important for many cell activities, including cell growth and division (proliferation), movement (migration) of cells, production of new proteins, transport of materials within cells, and cell survival. Studies suggest that PI3K signaling may also be involved in the regulation of several hormones and may even play a role in the maturation of fat cells (adipocytes).

In some embodiments, the methods of treating a cancer provided herein comprise treating a cancer comprising a loss of function mutation in a PI3K gene or a deletion of a PI3K gene. In some embodiments, the method further comprises determining if the patient (e.g., a cancer cell or tumor of the patient) has a loss of function mutation in a PI3K gene or a deletion of a PI3K gene. In some embodiments, the methods of treating a cancer provided herein comprise treating a cancer comprising a loss of function mutation in a Phosphatidylinositol-4,5-Bisphosphate 3-Kinase Catalytic Subunit Alpha (“PIK3CA”) gene or a deletion of a PIK3CA gene. A PIK3CA mutation is a genetic alteration in the PIK3CA gene that may result in a change in the protein sequence.

The PIK3CA gene may be an important gene in the PI3K pathway, as it may provide instructions for making the p110 alpha (p110α) protein, which is one piece (subunit) of the PI3K enzyme. The p110α protein, sometimes called the catalytic subunit, performs the action of PI3K, while the other subunit (produced by a different gene) regulates the enzyme's activity. Therefore, the PIK3CA gene may be an important target in treating a cancer.

PIK3CA mutations have been identified in a variety of human cancers, including, but not limited to, breast, colon, ovarian, and endometrial cancers, and PIK3CA mutations are one of the most frequently mutated genes in human cancers.

PI3K signaling may be important for normal cell function and dysregulation of this pathway can contribute to cancer development and progression. PIK3CA mutations are one mechanism by which the PI3K/AKT/mTOR pathway can become dysregulated in cancer. Targeting this pathway with drugs that inhibit PI3K, AKT, or mTOR is an area of active research in cancer therapy, with a number of compounds targeting various nodes in the relevant pathway having been developed.

Although PI3K inhibitors provide a promising area of research in the study of cancer, the development of resistance to PI3K inhibitors remains a significant challenge. Many of the relevant compounds have been unsuccessful in patients due to high levels of toxicity or their inability to downregulate the PI3K pathway. Studies have shown that cancers can disregard the downregulation of the PI3K pathway by either maintaining or re-establishing activation of the targeted pathway or by inducing alternate signaling pathways. In fact, a number of studies have shown that short-term treatments can result in the activation of feedback loops that subsequently decrease overall response rates, and that chronic administration of these therapies may result in the establishment of a reservoir of slow cycling cells that eventually may acquire resistance-conferring genetic mutations. Accordingly, there is a need to identify and research means for overcoming resistance to PI3K inhibitors.

One strategy to overcome challenges associated with cancers associated with PIK3CA gene mutations or deletions may be through synthetic lethality. Since many cancer cells already have a mutated PIK3CA gene, a promising approach is to identify and then pharmacologically target a synthetically lethal partner of one of the mutated PIK3CA gene or deleted PIK3CA gene. Surprisingly, the current inventors have discovered that cancer cells possessing a PIK3CA loss-of-function mutation are unexpectedly susceptible to inhibition of PI3Kβ activity.

In some embodiments, the mutation (e.g., loss of function mutation) or the deletion is in a PIK3CA gene. In some embodiments, the mutation is in a PIK3CA gene. In some cases, the mutation of a PIK3CA gene results in development of a cancer. In some embodiments, the mutation is a loss of function mutation. In some embodiments, the deletion is of a PIK3CA gene.

In some embodiments, said cancer comprises a loss of function mutation in the gene. In some embodiments, the loss of function mutation is in a PIK3CA gene. As used herein, a loss of function mutation may refer to a mutation in a gene that results in lower function of the gene, a lower function of a product of the gene, or a lower production of a product of the gene (e.g., an amount of protein produced by the gene (e.g., PI3K-alpha)).

In some embodiments, the method of treating a cancer in a patient in need thereof comprises determining whether the patient has a mutation (e.g., LOF mutation) or deletion of a PIK3CA gene (e.g., by obtaining or having obtained a biological sample form the patient), and if the patient has the mutation or deletion of the PIK3CA gene, then administering a PI3K inhibitor (e.g., a PI3K-beta inhibitor). In some embodiments, said method further comprises performing or having performed a test (e.g., assay) on the biological sample to determine if the patient has the mutation (e.g., LOF mutation) or deletion of the PIK3CA gene.

In some embodiments, said cancer comprises a deletion of a gene. In some embodiments, said deletion is of a PIK3CA gene. In some embodiments, the deletion of the PIK3CA gene results in the cancer of the patient or is a result of the cancer.

In some embodiments, the mutation or the deletion is in a PIK3CA gene. In some cases, the mutation of a PIK3CA genes results in a reduced amount or function of PI3K-alpha (e.g., as compared to a wild-type amount or function of PI3K-alpha).

In some embodiments, the method of treating a cancer in a patient in need thereof comprises determining whether the patient has a reduced amount or function of PI3K-alpha (e.g., by obtaining or having obtained a biological sample form the patient), and if the patient has the reduced amount or function of PI3K-alpha, then administering a PI3K inhibitor (e.g., a PI3K-beta inhibitor). In some embodiments, said method further comprises performing or having performed a test (e.g., assay) on the biological sample to determine if the patient has the reduced amount or function of PI3K-alpha.

PTEN levels may be used as a proxy for PI3K function and for patient selection to treat with PI3K inhibitors (e.g., PI3K-beta inhibitors), given its established function of inhibiting PI3K signaling. In some embodiments, said subject comprises a reduced level or amount of PTEN. In some embodiments, said reduced level or amount of PTEN may correspond to a loss of function mutation or a deletion of a PIK3CA gene. In some embodiments, said reduced level or amount of PTEN may correspond to a reduced amount or function of PI3K-alpha (e.g., as compared to a wild-type amount or function of PI3K-alpha).

In some embodiments, the method of treating a cancer in a patient in need thereof comprises determining whether the patient has a reduced amount or function of PTEN (e.g., by obtaining or having obtained a biological sample form the patient), and if the patient has the reduced amount or function of PTEN, then administering a PI3K inhibitor (e.g., a PI3K-beta inhibitor). In some embodiments, said method further comprises performing or having performed a test (e.g., assay) on the biological sample to determine if the patient has the reduced amount or function of PTEN.

d. PI3K-Alpha

Provided herein, in some embodiments, is a method of treating a cancer in a patient comprising a reduced amount or function of PI3K-alpha. In some embodiments, the method further comprises determining if the patient (e.g., a cancer cell or tumor of the patient) has a reduced amount or function of PI3K-alpha. PI3K-alpha is a product of PIK3CA. In some embodiments, the loss of function mutation in a PIK3CA gene or deletion of said PIK3CA gene reduces an amount or function of PI3K-alpha. In some embodiments, the reduced amount or function of PI3K-alpha is a result of a mutation or deletion of a PIK3CA gene. In some embodiments, a cancer causes the reduced amount or function of PI3K-alpha. In some embodiments, the treatment is with a different medication (e.g., other than a PI3K beta inhibitor). In some embodiments, the different medication is administered before a PI3K beta inhibitor. In some embodiments, the patient (e.g., a cancer cell or tumor of the patient) has been previously determined as comprising a reduced amount or function of PI3K-alpha.

In some embodiments, said patient (e.g., a cancer cell or tumor of the patient) has a reduced amount or function of PI3k-alpha (e.g., as compared to a wild-type amount or function of PI3K-alpha). In some embodiments, said patient (e.g., a cancer cell or tumor of the patient) has been previously determined as comprising a reduced amount or function of PI3k-alpha (e.g., as compared to a wild-type amount or function of PI3K-alpha). In some embodiments, the reduced amount or function of PI3K-alpha is not a result of pharmacological inhibition. In some embodiments, the method comprises administration of a composition comprising a PI3K inhibitor. In some embodiments, the method further comprises treatment with a different (e.g., an additional) medication, wherein the different medication is not a PI3K-beta inhibitor. In some embodiments, the different medication is administered before the composition comprising a PI3K-beta inhibitor. In some embodiments, the different medication is administered before the PI3K beta inhibitor. In some embodiments, the different medication is administered after the PI3K beta inhibitor

In some embodiments, the cancer comprises a loss of function mutation or a deletion of a PIK3CA gene. In some embodiments, the method further comprises determining if the patient (e.g., a cancer cell or tumor of the patient) has a loss of function mutation or a deletion of a PIK3CA gene. In some embodiments, the mutation or a deletion of a PIK3CA gene (e.g., wherein said mutation or deletion of the PIK3CA gene causes or is a result of the cancer) causes a reduced amount or function of PI3K-alpha (e.g., as compared to a wild-type amount or function in the patient (e.g., a cancer cell or tumor of the patient)).

In some embodiments, the patient, or a cancer cell of the patient, has been determined to comprise an amount of PI3K-alpha that is reduced compared to a wild-type amount of PI3K-alpha. In some embodiments, the patient, or a cancer cell of the patient, has been determined to comprise an amount of PI3K-alpha that is reduced compared to the average amount of PI3K protein in tissue of origin-matched tumors with wild-type PI3K-alpha. In some embodiments, patient (e.g., a cancer cell of the patient) has been determined to comprise a reduced amount of PI3K-alpha function compared to a wild-type amount of PI3K-alpha function.

In some embodiments, the reduced amount or function of PI3K-alpha is a result of a mutation or deletion of a PIK3CA gene (e.g., that causes or is caused by a cancer).

e. Types of Cancer

A loss of function mutation or deletion of a PIK3CA gene may result in a cancer or may exacerbate the severity or metastasis of the cancer. The methods provided herein may be useful in the treatment of a cancer, such as a cancer comprising a mutation (e.g., a loss of function mutation) in a PIK3CA gene or a deletion of a PIK3CA gene. These methods may be useful in the treatment of cancer, such as a cancer comprising a reduced amount of PI3K-alpha or function of PI3K-alpha.

Evidence for a PIK3CA loss of function mutation may be manifested as reduced PI3K signaling. Evidence has demonstrated that a loss of function in a PIK3CA gene is particularly strong in breast cancer, urothelial cancer (e.g., bladder cancer), rectal cancer, thymus cancer, sarcoma, endometrial, skin, bladder, stomach, and head and neck cancer.

In some embodiments, the cancer to be treated comprises breast cancer, urothelial cancer, rectal cancer, thymus cancer, sarcoma, or a combination thereof. In some embodiments, the cancer comprises bladder cancer, rectal cancer, breast cancer, a sarcoma, thymus cancer, or a combination thereof. In some embodiments, the cancer comprises bladder cancer. In some embodiments, the cancer comprises rectal cancer. In some embodiments, the cancer comprises breast cancer. In some embodiments, the cancer comprises a sarcoma. In some embodiments, the cancer comprises thymus cancer. In some embodiments, the cancer comprises a pancreatic cancer.

In some embodiments, the cancer comprises urothelial cancer. In some embodiments, the urothelial cancer comprises bladder cancer. In some embodiments, the cancer comprises rectal cancer. In some embodiments, the cancer comprises thymus cancer. In some embodiments, the cancer comprises sarcoma.

f. Identification of the Mutation or Deletion of the Gene (e.g., PIK3CA Gene)

In some embodiments, the cancer has been determined (e.g., previously determined) to comprise the mutation or deletion (e.g., of the PIK3CA gene). The presence of the mutation of the gene or deletion of the gene may be determined before administration of the composition. For example, the determination (e.g., previous identification) of the mutation or the deletion may take place months, weeks, days, hours, or even minutes ahead of the administering of the composition (e.g., the composition comprising a PI3K inhibitor). The determination of the presence of the mutation (e.g., loss of function mutation) in the gene or deletion of the gene (e.g., the PIK3CA gene) may be conducted in any appropriate manner, including, but not limited to, identification of a gene mutation or gene deletion by any one or more tests discussed herein.

In some embodiments, the mutation or deletion of the PIK3CA gene is a cause or result of the cancer. In some embodiments, the mutation or deletion of the PIK3CA gene is not a result of pharmacological inhibition. For example, in such embodiments, the mutation or deletion of the PIK3CA gene is not caused by administration of a PI3K inhibitor (e.g., a PI3K alpha inhibitor).

In some embodiments, the cancer has been determined (e.g., previously determined) as comprising a reduced amount or function of PI3K-alpha. The presence of the reduced amount of function of PI3K-alpha may be determined before administration of the composition. For example, the identification of the reduced amount of function of PI3K-alpha may take place months, weeks, days, hours, or even minutes ahead (i.e., previously identified) of the administering of the composition (e.g., the composition comprising a PI3K inhibitor). Determination of the presence of the reduced amount of PI3K-alpha or function of PI3K-alpha may be conducted in any appropriate manner, including, but not limited to, identification by of any one or more tests discussed herein.

In some embodiments, the reduced amount or function of PI3K-alpha is a cause or result of the cancer. In some embodiments, the reduced amount or function of PI3K-alpha is not a result of pharmacological inhibition. For example, in such embodiments, the reduced amount or function of PI3K-alpha is not caused by administration of a PI3K inhibitor (e.g., a PI3K alpha inhibitor).

The mutation (e.g., loss of function mutation or deletion in or of a PIK3CA gene) may be any acceptable type of mutation, such as, for example, a substitution mutation, an insertion mutation, a deletion mutation, or a combination thereof. Determination of whether the mutation or deletion of the PIK3CA gene may be used to determine whether a patient comprises a reduced amount or function of PI3K-alpha as compared to a wild-type amount or function of PI3K-alpha. In some embodiments, the PIK3CA mutation is a substitution mutation. In some embodiments, the PIK3CA mutation is an insertion mutation. In some embodiments, the PIK3CA mutation is a deletion mutation.

Determining if a tumor has a PIK3CA genetic mutation may include identifying PIK3CA mutations in DNA extracted from a tumor sample and/or in circulating tumor or tumor cell DNA. In some embodiments, the cancer is determined to comprise the mutation before administration of the PI3K inhibitor (i.e., the cancer is previously determined as comprising the mutation).

Multiple tests may be employed to detect PIK3CA mutations or deletions. For example, tests may include sequencing of the tumor DNA using Sanger sequencing of PCR-amplified PIK3CA encoding regions or next-gen sequencing (NGS) of whole genome or captured/enriched PIK3CA encoding regions (e.g., through whole-exome sequencing, or sequencing of a targeted mutation panel that includes PIK3CA exons and introns).

Mutations may also be detected in RNA. Such RNA mutations may be detected using RNA-Seq or DNA approaches to sequence cDNA derived from RNA. Furthermore, mutations may be detected through targeted amplification of variants and sequenced by next-generation sequencing (NGS) or through array-based readout of genetic variants (e.g., using Illumina BeadArrays). Tumor DNA may be derived from biopsy samples or captured from indirect tumor sources, such as blood derived cell-free DNA.

Function and amount of PI3K or PI3K-alpha may be determined through any acceptable method, including, for example, methods that measure the phosphorylation levels of known PI3K signaling components. Methods to quantify these modifications may include western blots, mass-spectrometry, protein binding arrays, or immunohistochemistry. Some examples of markers that may be used for measuring function and amounts of PI3K or PI3K-alpha include, but are not limited to AKTpS473 (phosphorylation of serine amino acid position 473 of protein AKT), GSK3 alpha/beta pS21/pS9, GSK3 pS9, Tuberin p1462, or a combination thereof.

g. Improving Cancer Treatment Efficacy

Methods provided herein may be useful in treating a cancer, such as, for example, by increasing cancer cell inhibition or decreasing cancer progression. “Increases cancer cell inhibition” may mean decreasing cancer cell viability, inhibiting cancer cell growth or proliferation, or killing of cancer cells.

In some embodiments, the administering of a PI3K beta inhibitor increases cancer cell inhibition. In some embodiments, the administering of a PI3K beta inhibitor increases cancer cell inhibition as compared to a different treatment. In some embodiments, administering of a PI3K beta inhibitor (e.g., selective PI3K beta inhibitor) increases cancer cell inhibition. In some embodiments, the different treatment comprises a pan-PI3K inhibitor, a PI3K-alpha inhibitor, a PI3K-gamma inhibitor, or a PI3K-delta inhibitor. In some embodiments, administering of a PI3K beta inhibitor increases cancer cell inhibition in a cancer comprising a loss of function mutation or a deletion of a PIK3CA gene compared to a corresponding administration of said PI3K beta inhibitor to a second cancer that does not have a loss of function mutation or a deletion of a PIK3CA gene. In some embodiments, administering of a PI3K beta inhibitor increases cancer cell inhibition in a cancer comprising a reduced amount or function of PI3K-alpha compared to a corresponding administration of said PI3K beta inhibitor to a second cancer that does not have a reduced amount or function of PI3K-alpha.

h. Administration

Administration of the composition comprising a PI3K inhibitor may be accomplished by any acceptable means, including, for example, by parenteral administration. Acceptable means of parenteral administration include, but are not limited to, subcutaneous administration, intramuscular administration, and intravenous administration. In some embodiments, the method comprises administering a PI3K inhibitor (e.g., a PI3K beta inhibitor). In some embodiments, administering comprises administering the PI3K inhibitor parenterally. In some embodiments, the method comprises administering a pharmaceutical composition comprising a PI3K inhibitor (e.g., such as any of the pharmaceutical compositions provided herein).

In some embodiments, administering comprises administering the PI3K inhibitor enterally. Acceptable means of enteral administration include, but are not limited to, oral administration, gastric administration, and rectal administration.

In some embodiments, administering occurs once daily. In some embodiments, administering occurs twice daily. In some embodiments, administering occurs three times daily.

i. Pharmaceutical Compositions

The PI3K inhibitor may be delivered in the form of a pharmaceutical composition. In some embodiments, the pharmaceutical composition comprises a PI3K inhibitor (e.g., AZD-8186 or GSK-2636771). The pharmaceutical composition may be useful in the treatment of a cancer, such as, for example, a cancer determined as comprising a loss of function mutation or a deletion of a PIK3CA gene or a cancer determined as comprising a reduced amount or function of PI3K-alpha as compared to a wild-type amount or function of PI3K-alpha.

The compositions described herein may comprise any appropriate PI3K inhibitor, such as, for example, any of the PI3K inhibitors described herein (e.g., a PI3K-beta inhibitor). In some embodiments, the PI3K inhibitor is a stereoisomer, pharmaceutically acceptable salt, hydrate, or solvate of a PI3K inhibitor described herein. In some embodiments, the pharmaceutical composition comprises a PI3K inhibitor and a pharmaceutically acceptable carrier (e.g., one or more pharmaceutically acceptable carrier, two or more pharmaceutically acceptable carriers, three or more pharmaceutically acceptable carriers, etc.). The carrier(s) may be any acceptable or suitable described herein. In some embodiments, the pharmaceutical composition comprising a PI3K inhibitor is administered in a method of treating a patient (e.g., comprising a PIK3CA loss of function mutation or a deletion of PIK3CA).

In certain embodiments, the PI3K inhibitor described herein is administered as a pure chemical (i.e., not with an excipient). In some embodiments, the PI3K inhibitor described herein is combined with a pharmaceutically suitable or acceptable carrier (which may also be referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

In some embodiments, the pharmaceutical composition comprising a PI3K inhibitor is formulated for oral administration. Suitable oral dosage forms include, but are not limited to, tablets, pills, sachets, or capsules.

In some embodiments, the pharmaceutical composition comprising a PI3K inhibitor is formulated for administration by injection. In some instances, the injection formulation is an aqueous formulation. In some instances, the injection formulation is a non-aqueous formulation.

In some instances, the injection formulation is an oil-based formulation, such as sesame oil, or the like.

The dose of the composition comprising a PI3K inhibitor may differ depending upon the subject or patient's condition. Such factors for consideration may include general health status, age, and other factors.

The pharmaceutical compositions described herein may be administered in any manner appropriate for the treatment or prevention of the disease (e.g., cancer). An appropriate dose and a suitable duration and frequency of administration may be determined by factors related to the condition of the patient, such as the type and severity of the patient's disease, the patient's age, weight, body surface area, etc. Alternatively, an appropriate dose and a suitable duration and frequency of administration may be determined by factors related to the composition, such as the particular form of the active ingredient, and the method of administration. In cases, the appropriate dose is determined by factors related to both the condition of the patient and the composition. In general, an appropriate dose and treatment regimen may provide the composition(s) in an amount sufficient to provide therapeutic and/or prophylactic benefit (e.g., an improved clinical outcome, such as more frequent complete or partial remissions, or longer disease-free and/or overall survival). Optimal doses are generally determined using experimental models and/or clinical trials. The optimal dose may depend upon the body mass, weight, or blood volume of the patient. In some embodiments the dose of GSK-2636771 is 100 mg once per day.

### II. Kits for Treating Cancer

Further provided herein, in some embodiments, are kits for treating a cancer, comprising: a test for determining: a loss of function mutation or a deletion of a PIK3CA gene or a reduced amount or function of PI3K-alpha as compared to a wild-type amount or function of PI3K-alpha; and a phosphoinositide 3-kinase (“PI3K”) inhibitor. In some embodiments, said PI3K inhibitor is a PI3K-beta inhibitor. In some embodiments, said PI3K-beta inhibitor is GSK-2636771, AZD-6482, AZD-8186, KA-2237, SAR-260301, or a combination thereof. In some embodiments, said PI3K-beta inhibitor is AZD8186. In some embodiments, said PI3K-beta inhibitor is a selective PI3K-beta inhibitor. In some embodiments, said selective PI3K-beta inhibitor is GSK-2636771, AZD-6482, AZD-8186, KA-2237, or SAR-260301. In some embodiments, said selective PI3K-beta inhibitor is GSK-2636771. In some embodiments, said selective PI3K-beta inhibitor is AZD-6482. In some embodiments, said selective PI3K-beta inhibitor is SAR-260301. In some embodiments, said selective PI3K-beta inhibitor is AZD-8186. In some embodiments, said selective PI3K-beta inhibitor is KA-2237.

In some embodiments, said cancer comprises breast cancer, urothelial cancer, rectal cancer, thymus cancer, sarcoma, or a combination thereof. In some embodiments, said urothelial cancer comprises bladder cancer.

### Definitions

As used in the specification and appended claims, unless specified to the contrary, the following terms have the meaning indicated below.

The singular forms (e.g., “a,” “and” and “the”) include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included.

The term “about” when referring to a number or a numerical range means that the number or numerical range referred to is an approximation within experimental variability (or within statistical experimental error), and thus the number or numerical range, in some instances, the term “about” preceding a stated value includes the stated value and also includes ±20% of the stated value or range, and includes more specifically values of ±10%, ±5%, ±2%, and ±1% of the stated value or range.

The term “associated” or “associated with” in the context of a disease, substance or substance activity or function associated with a disease means that the disease is caused by (in whole or in part), a symptom of the disease is caused by (in whole or in part) the substance or substance activity or function, or a side-effect of the compound (e.g., toxicity) is caused by (in whole or in part) the substance or substance activity or function.

The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

“Disease” or “condition” refer to a state of being or health status of a patient or subject capable of being treated with the compounds or methods provided herein. In some embodiments, the disease as used herein refers to cancer.

As defined herein, the terms “inhibition”, “inhibit”, “inhibiting” and the like, when made in reference to a protein-inhibitor interaction, means negatively affecting (e.g., decreasing) the activity or function of the protein (e.g., PI3K) relative to the activity or function of the protein in the absence of the inhibitor. The protein may be PI3K, such as, for example, PI3K-beta. Thus, inhibition includes, at least in part, partially or totally blocking stimulation, decreasing, preventing, or delaying activation, or inactivating, desensitizing, or down-regulating signal transduction or enzymatic activity or the amount of a protein. Inhibition, and such terms, may refer to reduction of a disease or symptoms of disease when referred to in such context.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

As used herein, “delaying development of a disease” means to defer, hinder, slow, retard, stabilize, and/or postpone development of the disease (such as cancer). This delay can be of varying lengths of time, depending on the history of the disease and/or individual being treated.

As is evident to one skilled in the art, a sufficient or significant delay can, in effect, encompass prevention, in that the individual does not develop the disease. For example, a late stage cancer, such as development of metastasis, may be delayed.

The terms “selective” or “selectivity” “specific”, “specifically”, “specificity”, or the like of a compound refers to the compound's ability to cause a particular action or the compound's ability to discriminate between molecular targets, such as inhibition, to a particular molecular target (e.g., PI3K-beta). For example, a selective PI3K-beta inhibitor may have at least a 3× higher selectivity for the PI3K-beta isoform compared to other isoforms of PI3K (e.g., PI3K-alpha, PI3K-gamma, or PI3K-delta) in a cell.

As used herein, compounds providing inhibitory properties (e.g., a PI3K inhibitor), include small-molecule compounds and biological products (e.g., such as those derived from living material (e.g., antibodies, proteins, peptide fragments, etc), unless context clearly dictates otherwise.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

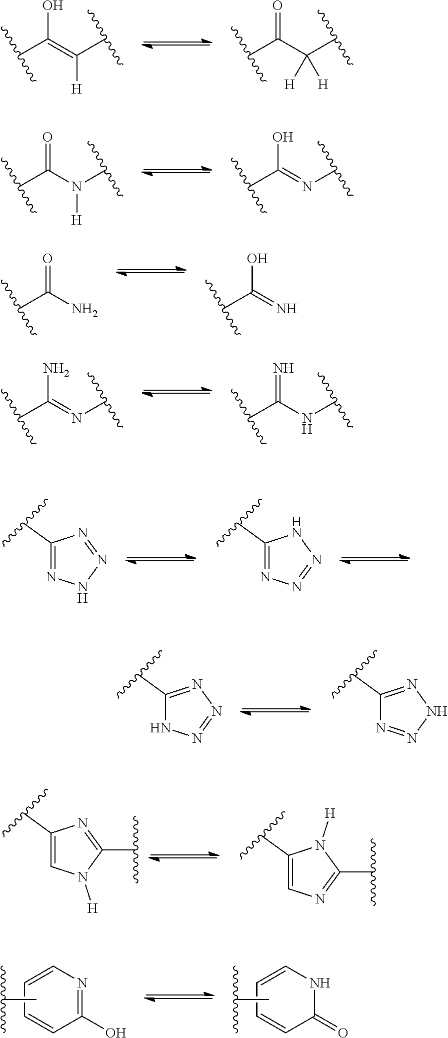

“Pharmaceutically acceptable salt” includes both acid and base addition salts. A pharmaceutically acceptable salt of any one of the PI3K inhibitors described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Pharmaceutically acceptable solvate” refers to a composition of matter that is the solvent addition form. In some embodiments, solvates contain either stoichiometric or non-stoichiometric amounts of a solvent, and are formed during the process of making with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. The compounds provided herein exist in either unsolvated or solvated forms.

The term “subject”, “individual” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

As used herein, “mutations” may refer to changes in a polynucleotide sequence that result in changes to protein function. A change to protein function may occur through a change to the protein encoded by the polynucleotide sequence. The change in function of the protein may be altered through an amino acid sequence of the protein or by a change in the amount (e.g., in an expression level) of the protein, or both. Mutations can be nucleotide substitutions, such as single nucleotide substitutions, insertions, or deletions, and may end up altering the splicing of messenger RNA (mRNA), the levels of mRNA, and/or the amino acid sequence of the protein encoded by said mRNA.

As used herein, “treatment” or “treating,” or “palliating” or “ameliorating” are used interchangeably. These terms refer to an approach for obtaining beneficial or desired results, including, but not limited to, a therapeutic benefit and/or a prophylactic benefit. The term “therapeutic benefit”, or the like, may include eradication or amelioration of the underlying disorder being treated. Also, a therapeutic benefit may be achieved with the eradication or amelioration of one or more of the symptoms associated with the underlying disorder such that an improvement may be observed in the patient, notwithstanding that the patient may still afflicted with the underlying disorder. For achieving a prophylactic benefit, the compositions may be administered to a patient at risk of developing a particular disease, or to a patient reporting one or more of the physiological symptoms of a disease, even though a diagnosis of this disease has not been made.

### EXAMPLES

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed in practicing the invention. It is intended that the following claims define the scope of the invention and that methods and structures within the scope of these claims and their equivalents be covered thereby.

### Example 1-Identification of Mutations in PIK3CA Gene

Experiments were run in order to identify potential loss of function mutations and deletions in PIK3CA. Genomic DNA was extracted from FFPE-preserved tumor biopsy tissue. Targeted capture was used to enrich for the exome, including all coding exons of all PIK3CA isoforms. NGS libraries were constructed and indexed using Illumina TruSeq exome kits (cat number 20020614) and then sequenced on an Illumina HiSeq platform. Somatic mutations were then called (e.g., per methods described in PMID 24192750 or PMID 33106175). Genome variants (SNVs and indels) were identified using GATK (PMID 20644199), and somatic mutations were called using Mutect2 (PMID 23396013) and Varscan (PMID 19542151). The mutations were filtered to functional/deleterious mutations on the basis of causing amino acid substitutions or protein coding changes with a predicted functional effect inferred by MutationTaster (PMID 20676075), a “deleterious” flag inferred by SIFT (PMID 12824425), and/or a “probably damaging” flag inferred by Polyphen-2 (PMID 23315928). Loss of function (LOF) mutations were filtered from likely gain of function (GOF) variants based on the absence of hotspot variants in LOF mutations. For example, LOF mutations may be identified as those that do not result, for example, in substitutions/deletions/alterations among the commonly altered PIK3CA amino acids. Commonly altered PIK3CA amino acids may include 1, 38, 39, 81, 88, 90, 93, 104, 106, 107, 108, 111, 115, 118, 344, 345, 350, 357, 364, 365, 378, 420, 453, 471, 539, 542, 545, 546, 604, 726, 901, 939, 970, 1004, 1007, 1021, 1025, 1043, 1044, 1047, 1049, 1052, 1065, or a combination thereof. For example, one possible hotspot mutation for amino acid 545 is the mutation E545K, which is a change of glutamic acid at position 545 to lysine.

Deep deletions (e.g., which may include a complete (zero copy) loss of one or more regions of PIK3CA that overlap a portion of a coding exon or an entire coding exon) may also constitute PIK3CA LOF events. Deep deletions and a complete loss of the PIK3CA gene were called from exome data using Control-FREEC (PMID 22155870). Western analysis and immunohistochemistry on FFPE preserved tumor biopsy was used to confirm a reduction or loss of PIK3CA gene expression at the protein level.

### Example 2—Analysis of PIK3CA Loss of Function or Deletion in Patients with Tumors Harboring PIK3CA Mutations

Precision medicine may provide some of the newest approaches to cancer treatment. Targeted therapeutics may be designed to exploit disease biology and to define which patients should receive a specific therapy. One concept underlying this approach is that the molecular features of a patient's tumor may influence clinical response to a medication, and thus, may be used to guide therapy (e.g., medication selection). As such, precision medicine may lead to increased efficacy (e.g., increased tumor cell growth inhibition or death) and reduced toxicity.

Tumor genetics may be central to this approach. For example, cancer may be caused by driver mutations, which confer a selective advantage and may allow cancer cells to proliferate. Driver mutations may be used to define patients and may be highly predictive for drug activity; that is, the presence of a specific driver mutation may be an indicator of the effectiveness of a given therapy. Approximately one third of cancers may be caused by driver mutations that induce a gain of function (GOF) in oncogenes and result in a protein product that may be therapeutically targeted. The remaining two thirds of cancers are caused by a loss of function (LOF) mutation in a tumor suppressor gene (TSG). In healthy cells, TSGs may act to control cell growth and division; loss of function mutations in these genes may result in uncontrolled growth and cancer. Such cancers are typically more difficult to target than cancers resulting from GOF mutations, because in cancers resulting from a LOF mutation it is the absence of a functional protein that results in oncogenesis. Thus, a target is more difficult to ascertain. As evidence of this disparity in treatability, drugs exist against approximately 2/3 of oncogenes, but drugs exist for only ~2% of tumor suppressor genes (e.g., the PARPi targeting homologous recombination defects, e.g., BRCA1/2, ATM, ATR, mutations). One potential approach to targeting LOF cancers is to leverage vulnerabilities that are unique to tumor cells by identifying synthetic lethal (SL) interactions. In a synthetic lethality relationship between two genes a loss of function in either, but not both, is tolerated. Since only tumor cells contain the driver mutation, drugs that specifically target the mutation will only impact these tumor cells, and off-target effects, such as toxicities, are expected to be minimal. This approach may be applied to identify both genetic dependencies and small molecule sensitivities of cancer cell lines. As evidence of the importance of this approach, massive and ongoing efforts by large consortia are underway to comprehensively map synthetic lethal networks in hundreds of cancer cell lines with the goal of identifying drug targets and the patients who are expected to respond best to them.

While these efforts have provided valuable insights and identified potential drug candidates, significant challenges remain. For example, many patients do not benefit from targeted therapies because large-scale screens produce new target predictions and feed drug development pipelines at the entry point such that treatment is years away, and the drug(s) identified in screens (e.g., small molecule screens) may not recapitulate genetic perturbation profiles and SL relationships identified in cell lines may not translate to tumors in patients.

Using data from The Cancer Genome Atlas (“TCGA”; www.cancer.gov/tcga and PMID 28528867) the effect of PIK3CA LOF mutations on PI3K pathway activity was tested. First, the prevalence of PIK3CA LOF mutations/deletions in multiple cancers was evaluated. From data gathered from TCGA, subjects were organized into three categories for each ofthe cancer types captured by TCGA: 1) those comprising a PIK3CA LOF mutation (e.g., such as using the mutation calling criteria described in Example 1), 2) those comprising a wild-type (wt) PIK3CA, or 3) those comprising a PIK3CA GOF mutation (e.g., comprising a hotspot mutation or more than 2 copies of PIK3CA). The proportion of subjects with a PIK3CA LOF mutation/deletion was then evaluated for each ofthe respective cancers, the results ofwhich are summarized in Table 1. As demonstrated by Table 1, a large proportion of patients with different types of cancers comprise a PTK3CA LOF mutation or deletion.

TABLE 1
Cancer types with loss of function PIK3CA tumors
and their estimated prevalence from TCGA.

| | | Patients |
|---|---|---|
| | | with LOF |
| Cancer Type | TCGA Cancer ID | PIK3CA (%) |
| Uterine Corpus Endometrial | TCGA-UCEC | 11.30% |
| Carcinoma | | |
| Bladder Urothelial Carcinoma | TCGA-BLCA | 4.35% |
| Skin Cutaneous Melanoma | TCGA-SKCM | 4.31% |
| Uterine Carcinosarcoma | TCGA-UCS | 3.77% |
| Rectum Adenocarcinoma | TCGA-READ | 3.33% |
| Colon Adenocarcinoma | TCGA-COAD | 2.87% |
| Cervical Squamous Cell Carcinoma | TCGA-CESC | 2.37% |
| and Endocervical Adenocarcinoma | | |
| Stomach Adenocarcinoma | TCGA-STAD | 2.30% |
| Breast Invasive Carcinoma | TCGA-BRCA | 2.06% |
| Head and Neck Squamous Cell | TCGA-HNSC | 2.02% |
| Carcinoma | | |
| Kidney Chromophobe | TCGA-KICH | 1.52% |
| Glioblastoma Multiforme | TCGA-GBM | 1.45% |
| Lung Adenocarcinoma | TCGA-LUAD | 1.39% |
| Lung Squamous Cell Carcinoma | TCGA-LUSC | 1.27% |
| Uveal Melanoma | TCGA-UVM | 1.25% |
| Sarcoma | TCGA-SARC | 1.22% |
| Mesothelioma | TCGA-MESO | 1.22% |
| Pancreatic Adenocarcinoma | TCGA-PAAD | 1.12% |
| Brain Lower Grade Glioma | TCGA-LGG | 1.00% |
| Kidney Renal Clear Cell Carcinoma | TCGA-KIRC | 0.63% |
| Thyroid Carcinoma | TCGA-THCA | 0.62% |
| Pheochromocytoma and | TCGA-PCPG | 0.62% |
| Paraganglioma | | |
| Esophageal Carcinoma | TCGA-ESCA | 0.55% |
| Liver Hepatocellular Carcinoma | TCGA-LIHC | 0.54% |
| Ovarian Serous Cystadenocarcinoma | TCGA-OV | 0.49% |
| Prostate Adenocarcinoma | TCGA-PRAD | 0.41% |

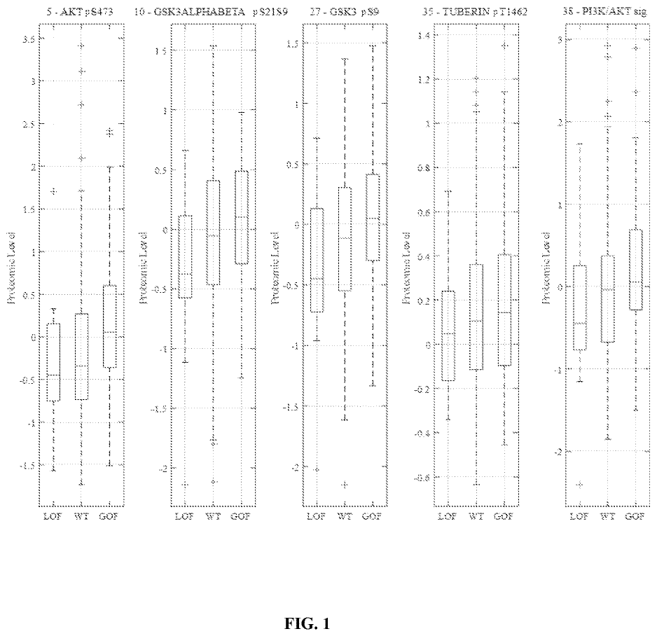

Next, the correlation between PIK3CA mutation status and PI3K activity was evaluated. GSK3 alpha/beta pS21/pS9, GSK3 pS9, and Tuberin p1462 are phosphorylation markers that may reflect PI3K activity. The distribution of these phosphorylation markers and a global signal of activity described by Zhang et al. 2017 (PMID 28528867) were compared. This analysis found a correlation between PIK3CA mutation status and PI3K activity. In particular, a strong correlation between PIK3CA mutations and PI3K activity was found in bladder (BLCA), rectal (READ), breast (BRCA, including TNBC), Sarcoma (SARC), and Thymus (THYM) cancer. Results of the comparative analysis in breast cancer cells are shown in FIG. 1.

As demonstrated by the above analysis, PIK3CA LOF mutations exist in a meaningful number of patients (see, e.g., Table 1) and are associated with reduced PI3K activity.

### Example 3—A Study of PI3K Inhibitors for the Treatment of Tumors Harboring PIK3CA Mutations

To determine the effects of PI3K inhibitors in tumor cells harboring PIKCA mutations, cell lines with engineered PIK3CA were created and administered multiple PI3K inhibitors. In particular, an engineered line comprising a PIK3CA loss of function (LOF) mutant was compared to its parental line (PIK3CA wild type), and a cell line with PIK3CB knocked out. Various PI3K inhibitors were administered to each cell line.

To create cells with a loss of function PIK3CA mutation (PIK3CA-LOF mutant), the PIK3CA gene was knocked out in KP4 cells using CRISPR genome editing (kits sold by Synthego). The parental line of KP4 cells comprising PIK3CA wild type was used as a comparator, and KP4 cells with PIK3CB knocked out were used as a control. A cell viability assay was performed by treating the three isogenic lines with various concentrations of three different PI3K inhibitors: GSK-2636771, a selective PI3K-beta (PIK3CB) inhibitor, AZD-8186, a PI3K-beta/delta (PIK3CB, PIK3CD) inhibitor, and seletalisib, a selective PI3K-delta (PIK3CD) inhibitor.

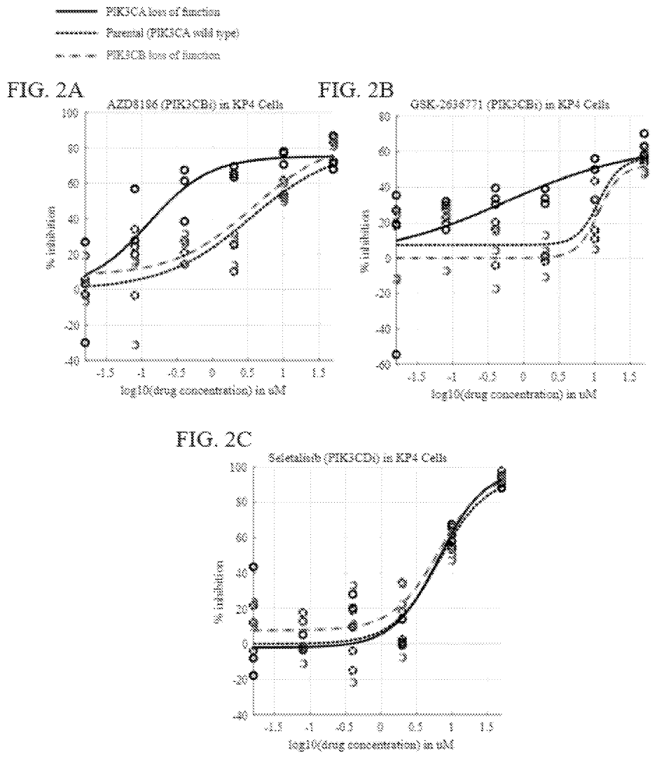

As shown in FIG. 2, cells having a PIK3CA LOF mutation exhibited increased sensitivity to PI3K-beta inhibitors, such as GSK-2636771 and AZD-8186. However, no such effect was observed with seletalisib, a PI3K-delta inhibitor. FIG. 2A demonstrates favorable increases in cancer cell inhibition when AZD-8186, a PI3K inhibitor that potently inhibits PI3Kβ and PI3Kδ, is administered to KP4 cells comprising a PIK3CA LOF mutation, compared to administration of AZD-8186 in KP4 cells comprising PIK3CA wild type or PIK3CB LOF. FIG. 2B demonstrates favorable increases in cancer cell inhibition when GSK-2636771, a selective PI3Kβ inhibitor, is administered to KP4 cells comprising a PIK3CA LOF mutation, compared administration of GSK-2636771 to KP4 cells comprising PIK3CA wild type or PIK3CB LOF. FIG. 2C demonstrates no improvement in cancer cell inhibition when seletalisib, a potent and selective PI3Kδ inhibitor, is administered to KP4 cells comprising a PIK3CA LOF mutation when compared to an administration of seletalisib to KP4 cells comprising PIK3CA wild type or PIK3CB LOF.

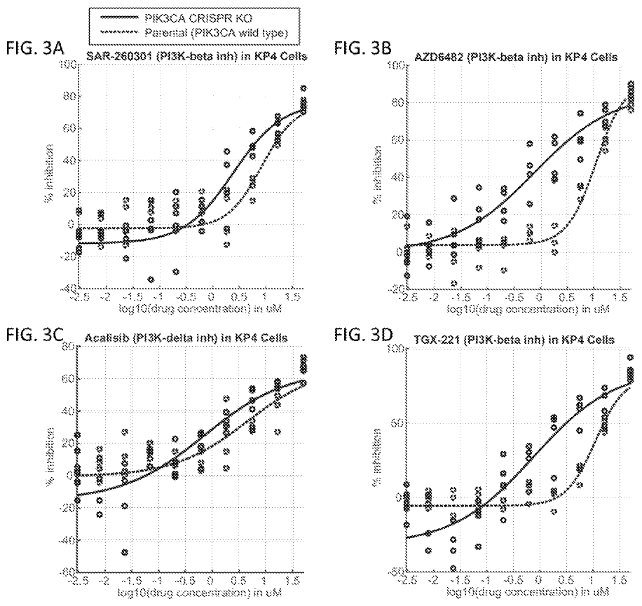

As shown in FIG. 3, cells having a PIK3CA LOF mutation exhibited increased sensitivity to PI3K-beta inhibitors, such as SAR-260301, AZD-6482, and TGX-221. However, no such effect was observed with Acalisib, aPI3K-delta inhibitor. FIG. 3A demonstrates favorable increases in cancer cell inhibition when SAR-260301, a selective PI3Kβ, is administered to KP4 cells comprising a PIK3CA LOF mutation or deletion, compared to administration of SAR-260301 in KP4 cells comprising PIK3CA wild type. FIG. 3B demonstrates favorable increases in cancer cell inhibition when AZD-6482, a selective PI3Kβ inhibitor, is administered to KP4 cells comprising a PIK3CA LOF mutation, compared administration of AZD-6482 to KP4 cells comprising PIK3CA wild type. FIG. 3C demonstrates reduced levels of cancer cell inhibition when Acalisib, a selective PI3Kδ inhibitor, is administered to KP4 cells comprising a PIK3CA LOF mutation when compared to an administration of Acalisib to KP4 cells comprising PIK3CA wild type. FIG. 3D demonstrates a favorable increase in cancer cell inhibition when TGX-221, a selective PI3Kβ inhibitor, is administered to KP4 cells comprising a PIK3CA LOF mutation when compared to an administration of TGX-221 to KP4 cells comprising PIK3CA wild type.

The results of this experiment demonstrate that there is synthetic lethality between PI3K-beta inhibitors and a PIK3CA loss of function mutation; however, no synthetic lethality exists between PI3K-delta inhibitors and a PIK3CA loss of function mutation. This experiment also demonstrated that inhibitors with PI3K-beta inhibition that also inhibit other PI3K variants (e.g., AZD-8186), may be useful in treating patients with a cancer comprising a PIK3CA LOF mutation.

### Example 4. Biochemical Assay

To determine the status of PI3K/Akt/GSK-3beta-mediated signal transduction in tumor samples, expression of PI3K-alpha, PI3K-beta, phosphorylated Akt (pSer473), phosphorylated GSK-3beta (pSer21/9), and PTEN are examined using immunohistochemistry. Furthermore, DNA is extracted from the tumor samples and assayed by exome sequencing and RNAseq, using methods, such as those set forth in Example 1.

The analysis will demonstrate a strong association between reduced levels of PI3K-alpha and LOF PIK3CA mutations, as well as an association between reduced PI3K-alpha protein levels and Akt pS473 and GSK-3beta pS21/9.

Results will demonstrate that PTEN negatively regulates the PI3K signaling pathway by dephosphorylating PIP3 to PIP2. When PTEN levels are low, as in the case of PTEN loss or mutation, PIP3 levels will increase, leading to the activation of Akt and GSK-3beta.

The results of this analysis will be consistent with demonstrating that Akt phosphorylation, GSK-3 phosphorylation, PTEN mutation status, and PIK3CA mutation status being an indicator of PI3K pathway activation.

### Example 5. Cellular Assay

To study the effect of additional selective PI3K-beta inhibitors on cells with a PIK3CA LOF mutation, cells with and without PIK3CA LOF mutations are administered a selective PI3K-beta inhibitor.

Isogenic pairs of KP4 cell lines differing only in their PIK3CA mutation status (with and without PIK3CA loss of function) are generated. Synthego CRISPR Gene Knockout v2 kits are used to knock out PIK3CA in KP4 cells (where PIK3CA is wild type and present in 2 copies; PMID 23550210). The knockout of PIK3CA is confirmed by Sanger sequencing and Synthego analysis software. Cells in 96-well plates at 2000 cells/well are seeded in triplicate and treated with varying concentrations of PI3K-beta inhibitors spanning a 3× dilution gradient from 50 uM to sub-nanomolar concentrations. Five days after treatment initiation, cell viability is determined by cell titer glo.

The curves will reveal a dose-dependent response to PI3K-beta inhibitors, with higher concentrations leading to greater reductions in cell viability, and overall higher sensitivity in cells with PIK3CA knocked out (i.e., a lower concentration of drug would yield the same reduction of viability in PIK3CA-LOF vs PIK3CA wt).

### Example 6. Xenograft Study Using Cell Derived Xenograft Models

Heightened sensitivity to a PI3K-beta inhibitor (e.g., GSK-2636771) caused by loss of function of PIK3CA will be analyzed in vivo using cell derived xenograft (CDX) models with isogenic paired KP4 cells (with/without PIK3CA knocked out). Study design includes 30 mice, 15 for each treatment (mice with PIK3CA LOF and mice with PIK3CA wild type). 5 replicates across 3 groups will be utilized, with the mice receiving vehicle (no treatment), 3 mg/kg of a PI3K-beta inhibitor, or 10 mg/kg of a PI3K-beta inhibitors. Cells are implanted into immunocompromised mice and allowed the tumors to grow to a palpable size. Over 28 days of treatment, tumors are sized using calipers.

A larger reduction in tumor growth will be observed in both drug treated (3 mg/kg and 10 mg/kg) PIK3CALOF groups, compared to vehicle PIK3CALOF control and PIK3CAwild type treatment groups. Furthermore, there will be no significant weight loss at any dose of treatment.

### Example 7. Xenograft Study Using Patient Derived Xenograft Models

To investigate the potential for using PI3K-beta inhibitor (e.g., GSK-2636771) to treat tumors with PIK3CA-LOF, patient derived xenograft (PDX) models with predicted LOF mutations in PIK3CA are identified from the NCI Patient Derived Model Repository (PDMR) and at Jackson Labs (JAX). Analysis of whole exome sequencing data to identify LOF PIK3CA mutations are done using methods, such as those described in Example 1. GATK and Samtools are used to call likely somatic single nucleotide variants (SNVs) and indels, which are then filtered on high likelihood of inducing a protein functional change, and further distinguished from gain of function (GOF) mutations on not being in sequence or structural hotspot regions. Loss of function (LOF) status is further reinforced on the basis of reduced expression of PIK3CA relative to other PDX models of the same cancer type. A variant allele frequency of at least 0.4 is required (at least 40% of the sequence reads that overlap the mutation contained the mutation in their sequence). Three exemplary models that met the criteria included: 997537-175-T colon model (PDMR), 475296-252-R rectal model (PDMR), and TM01117 breast model (JAX).

PDX samples harboring a PIK3CA LOF mutation, selected using the criteria described above, are cut to a uniform size and implanted subcutaneously into bilateral flanks of 5-week old NSG mice sourced from JAX. For each study (i.e., each model), a 3×8 (3 treatments, 8 replicates) experimental design is employed. Tumors are measured every other day until tumor volume reached approximately 150 mm3. GSK-2636771 is administered at three doses (10 mg/kg and 3 mg/kg and vehicle control) once daily by oral gavage. Tumor sizes are recorded daily, and body weights are measured every 7 days to monitor for drug toxicity. Tumor volume is calculated using the following formula: tumor volume (mm3)=1/2 (W)2×(L). Mice are sacrificed once tumors reached 1000 mm3. Differential outcomes are determined through a log rank Kaplan-Meier survival analysis.

Mice treated with either dose of GSK-2636771 will achieve significantly improved survival outcomes with significantly reduced tumor growth.

### Example 8. Patient Selection

Tumor derived FFPE biopsy samples are used to identify subjects with a LOF of PIK3CA. Next-generation sequencing (NGS) of a targeted gene panel that includes all exons of PIK3CA are used to generate sequencing data. Mutations are called using methods such as those as described in Example 1. Subjects with an adjusted variant allele frequency (adjVAF) that accounts for normal tissue contribution in the biopsy are selected for treatment with a PI3K-beta inhibitor if their adjVAF exceeded 0.3, which indicates that the LOF mutation occurred relatively early in the development of the tumor.

The examples and embodiments described herein are for illustrative purposes only and various modifications or changes suggested to persons skilled in the art are to be included within the spirit and purview of this application and scope of the appended claims.

What is claimed is:

1. A method of treating a cancer in a patient in need thereof, said method comprising:
administering a composition comprising a phosphoinositide 3-kinase (“PI3K”) inhibitor,
wherein said cancer has been previously determined as comprising a loss of function mutation or a deletion of a PIK3CA gene.

2. The method of claim 1, wherein said loss of function or deletion of said PIK3CA gene reduces an amount or function of PI3K-alpha as compared to a wild-type amount or function of PI3K-alpha.

3. A method of treating a cancer in a patient in need thereof, said method comprising:
administering a composition comprising a phosphoinositide 3-kinase (“PI3K”) inhibitor
wherein said patient has a reduced amount or function of PI3K-alpha as compared to a wild-type amount or function of PI3K-alpha.

4. The method of claim 3, wherein a mutation or deletion in a gene reduces said amount of function of PI3K-alpha.

5. The method of claim 4, wherein said gene is a PIK3CA gene.

6. The method of claim 4 or 5, wherein said mutation is a loss of function or deletion of said gene.

7. The method of claim 3, wherein a cancer cell has said reduced amount or function of PI3K-alpha.

8. The method of anyone of claims 1-7, wherein said PI3K inhibitor is a PI3K-beta inhibitor.

9. The method of claim 8, wherein said PI3K-beta inhibitor is GSK-2636771, AZD-6482, AZD-8186, KA-2237, SAR-260301, TGX-221, or a combination thereof.

10. The method of claim 8, wherein said PI3K-beta inhibitor is a selective PI3K-beta inhibitor.

11. The method of claim 10, wherein said selective PI3K-beta inhibitor is GSK-2636771, AZD-6482, AZD-8186, KA-2237, TGX-221, or SAR-260301.

12. The method of claim 11, wherein said selective PI3K-beta inhibitor is GSK-2636771.

13. The method of claim 9, wherein said PI3K-beta inhibitor is AZD-6482.

14. The method of claim 9, wherein said PI3K-beta inhibitor is AZD-8186.

15. The method of claim 9, wherein said PI3K-beta inhibitor is KA-2237.

16. The method of claim 9, wherein said PI3K-beta inhibitor is SAR-260301.

17. The method of claim 9, wherein said PI3K-beta inhibitor is TGX-221.

18. The method of any one of claims 8-17, wherein said administering of said PI3K-beta inhibitor decreases an adverse effect or a side effect compared to a corresponding administering of a pan-PI3K inhibitor, a PI3K-alpha inhibitor, a PI3K-gamma inhibitor, or a PI3K-delta inhibitor to said cancer.

19. The method of any one of claims 8-18, wherein said administering increases cancer cell inhibition compared to a corresponding administration of said composition to a second patient comprising a wild-type amount or function of PI3K-alpha.

20. A method of treating a cancer in a patient in need thereof, said method comprising:
administering to said patient a composition comprising a PI3K-beta inhibitor, wherein when said composition is administered, cancer cell inhibition increases compared to a corresponding composition comprising a PI3K inhibitor other than said PI3K-beta inhibitor.

21. The method of claim 20, wherein said PI3K-beta inhibitor is GSK-2636771, AZD-6482, AZD-8186, KA-2237, TGX-221, or SAR-260301.

22. The method of claim 20, wherein said PI3K-beta inhibitor is a selective PI3K-beta inhibitor.

23. The method of claim 22, wherein selective PI3K-beta inhibitor is GSK-2636771, AZD-6482, AZD-8186, KA-2237, TGX-221, or SAR-260301.

24. The method of claim 20, wherein said PI3K-beta inhibitor is an inhibitor of PI3K-beta and PI3K-delta.

25. The method of claim 20, wherein said PI3K-beta inhibitor is AZD8186 or KA-2237.

26. The method of anyone of claims 1-25, wherein said cancer comprises breast cancer, urothelial cancer, rectal cancer, thymus cancer, sarcoma, pancreatic cancer, or a combination thereof.

27. The method of claim 26, wherein said urothelial cancer comprises bladder cancer.

28. A kit for treating a cancer, comprising:
a. a test for determining either:
i. a loss of function mutation or a deletion of a PIK3CA gene; or
ii. a reduced amount or function of PI3K-alpha as compared to a wild-type amount or function of PI3K-alpha; and
b. a phosphoinositide 3-kinase (“PI3K”) inhibitor.

29. The kit of claim 28, wherein said PI3K inhibitor is a PI3K-beta inhibitor.

30. The kit of claim 29, wherein said PI3K-beta inhibitor is GSK-2636771, AZD-6482, AZD-8186, KA-2237, SAR-260301, TGX-221, or a combination thereof.

31. The kit of claim 29, wherein said PI3K-beta inhibitor is a selective PI3K-beta inhibitor.

32. The kit of claim 31, wherein said selective PI3K-beta inhibitor is GSK-2636771, AZD-6482, AZD-8186, KA-2237, TGX-221, or SAR-260301.

33. The kit of anyone of claims 28-32, wherein said cancer comprises breast cancer, urothelial cancer, rectal cancer, thymus cancer, sarcoma, pancreatic cancer, or a combination thereof.

34. The kit of claim 33, wherein said urothelial cancer comprises bladder cancer.
